(19) United States
(12) Patent Application Publication
(10) Pub. No.: US 2008/0033399 A1
(43) Pub. Date: Feb. 7, 2008

(54) SOFT CANNULA

(75) Inventors: Marcel Hunn, Burgdorf (CH); Jurg Liniger, Ostermundigen (CH); Patrik Denoth, Munchenwiler (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(73) Assignee: Disetronic Licensing AG

(21) Appl. No.: 11/872,339

(22) Filed: Oct. 15, 2007

Related U.S. Application Data
(62) Division of application No. 10/679,950, filed on Oct. 6, 2003.
Application No. 10/679,950 is a continuation of application No. PCT/CH02/00187, filed on Apr. 3, 2002.

(30) Foreign Application Priority Data
Apr. 6, 2001 (DE) 101 17 286.9

(51) Int. Cl. A61M 25/00

(52) U.S. Cl. 604/523

(57) ABSTRACT

A cannula which increases in pliability during use, wherein prior to use the cannula includes at least one material of variable hardness or at least two materials of differing hardness, of which the variably harder or material having the greater hardness is yielded or dissolved during application.

### CROSS REFERENCE TO RELATED APPLICATION(S)

This application is a Divisional of U.S. patent application Ser. No. 10/679,950, filed Oct. 6, 2003, which is a Continuation of International Patent Application PCT/CH02/00187, filed on Apr. 3, 2002, which claims priority to German Application No. 101 17 286.9, filed on Apr. 6, 2001. The contents of which are all hereby incorporated by reference.

### BACKGROUND

The present invention relates to a cannula which increases in pliability during application or use, to its use in medical devices and procedures, and to medical devices comprising such cannulae.

Cannulae have various applications in the field of medicine, in particular for transcutaneous or subcutaneous applications. To this end, it is necessary for the cannulae to exhibit a sufficient rigidity and hardness, in order to penetrate the skin without problems and without significant damage to the surrounding tissue. In many applications, the cannulae also have to be able to puncture materials outside the human or animal body, such as for example a septum, without problems. This must not cause damage to the septum, compromising its functionality, even after numerous penetration processes. Cannulae are therefore mostly metallic hollow needles with a chamfered tip, which guarantees the required penetrative capacity. Plastic cannulae with a metal mandril are also known.

In addition, the cannula also functions during its application as a transport channel through which fluids (e.g., solutions of medical active agents) are supplied to the body or body fluids are removed (e.g., for diagnostic purposes). What is important here is that the cannula exhibits a certain flow cross-section over its entire length, during its application. Constrictions of the cannula due to mechanical stresses, e.g., kinking by the cannula during its application, impede the transport of fluid and can lead to an undesired increase in the hydrostatic pressure in the cannula. Metallic or metalliferous cannulae are therefore used, to ensure that the flow cross-section remains as unchanged as possible during application.

Cannulae are, among other things, a component of medical devices for transcutaneous and subcutaneous applications. Examples of these are syringes, infusion apparatus, perfusion apparatus and catheter heads for the aforesaid apparatus. The cannulae have areas of different functionality. While the pointed end guarantees its penetrative capacity, another area of the cannula—generally the other end of the cannula—establishes the connection with components of the medical device, such as for example a catheter.

In some applications of these medical devices, it can be advantageous if the cannula is applied such that its end which penetrates the skin is positioned as near as possible to the desired target location in the human or animal body. The cannula can thus be specifically positioned for example such that the cannula is guided through the vascular system of the body, e.g., through the veins. When using rigid metal or metalliferous cannulae, this inevitably leads to injuries to the vascular system or the tissue. Conversely, soft and highly pliable cannulae enable the cannula to be guided or moved flexibly, but do not exhibit the hardness required for penetrating without problems.

In some medical devices, for example a catheter head, a cannula has to penetrate both a septum and skin tissue. Since the cannula used consists of a hard and rigid material, it can only be moved in a straight line. This in turn means that for penetrating without problems, the puncture planes of the septum and the skin have to be arranged as parallel as possible. If this is not the case, catheter heads often use two cannulae, one of which punctures the septum and the other the skin tissue.

### SUMMARY

It is an object of the invention to provide a cannula which both enables the skin, a septum or comparable materials to be penetrated without problems and exhibits a sufficient pliability during its application, such that the cannula is flexible and can be moved in any direction in the human or animal body or in a medical device, even if this requires changes in direction. In this way, a flow cross-section is to be constantly provided over the entire length of the cannula which enables fluids to be transported.

The object is addressed by a cannula which increases in pliability during application, wherein said cannula prior to application comprises at least one material of variable hardness or at least two materials of differing hardness, of which said material having the greater hardness is yielded during application.

Furthermore, it is an object of the invention to provide medical devices for transcutaneous and subcutaneous applications, in particular a transcutaneous infusion set, a transcutaneous perfusion apparatus and a catheter head for the aforesaid apparatus, which comprise one or more of the cannulae.

This object is addressed by a cannula which increases in pliability during application, wherein said cannula prior to application comprises at least one material of variable hardness or at least two materials of differing hardness, of which said material having the greater hardness is yielded during application, for use in a transcutaneous infusion set, a transcutaneous perfusion set or a catheter head, and by a transcutaneous infusion set including such a cannula, a transcutaneous perfusion set including such a cannula and a catheter head for a transcutaneous infusion set in which such a cannula forms an infusing part of said catheter head.

In accordance with the invention, a cannula exhibits an increasing pliability during its application, wherein the cannula prior to application comprises at least one material of variable hardness or at least two materials of differing hardness, of which the material having the greater hardness is yielded during application.

The term “application” as used herein is to be understood such that it refers to every possible application or use of the cannula in accordance with the invention. Prior to its use, the cannula is in an initial state. When the cannula is used, e.g., to penetrate the skin or a septum, transport fluids, etc., it is in or substantially in the initial state. In its initial state, the cannula can be exposed to a different environment, can come into contact with different substances and materials, and can have different uses, than it can later during its continued use or application.

In accordance with the invention, the term “pliability” refers to the capacity of the cannula to change its shape under mechanical and/or thermal stress. This may, for example, be expressed by the fact that the cannula changes under mechanical stress from a linear shape to a bent or curved shape, or that the shape of the cross-section of the cannula changes, e.g., from circular to elliptical.

Increasing pliability therefore means that greater mechanical and/or thermal stresses are necessary to cause the same change in shape in a cannula in the initial state prior to application than in a cannula during application. The increase in pliability can progress over time in different ways during application. The increase can for example be continuous, until a terminal value for pliability is reached. The terminal value can also be reached in a short interval in time, to then remain constant.

The pliability of a cannula in accordance with the invention, in the initial state, is such that it is possible to penetrate the skin, a septum or other materials without problems and deformation, e.g., kinking, which would significantly reduce the flow cross-section of the cannula or even reduce it to zero in sections.

The change in pliability during application is such that, due to its flexible shape, the cannula can be diverted both in the body and in medical devices and can, in some embodiments, preferably be guided in any direction. The cannula in accordance with the invention can be guided along or inside vascular systems, e.g., veins, even if this requires the cannula to be repeatedly diverted and/or bent. Furthermore, the cannula can be moved along a curved line, such that it can penetrate materials whose puncture areas are unfavorably arranged, e.g., at right angles to each other. Even only a local pliability can be advantageous. The cannula can be elastically, inelastically, viscoelastically or plastically pliable; a combination of two or more of these properties is also possible.

The term “hardness” of a material refers in accordance with the invention to the resistance which the material offers against the intrusion of hard objects into its surface. Material hardness can be determined by way of common methods such as for example the Brinell or Rockwell hardness test or the Vickers or Knoop micro-hardness testing methods.

A material of varying hardness in accordance with the invention exhibits a particular initial hardness in an initial state prior to use or application, starting from which the hardness changes during application. In accordance with the invention, the at least two materials of differing hardness are materials which exhibit a differing initial hardness, wherein the material having the greater hardness is at least partially yielded during application. In this way, the material composition of the cannula also changes during application.

Preferably, the material of variable hardness is a material whose hardness decreases during application, i.e., the initial hardness of the material prior to application is greater than the hardness of the material at a later point in time, during application. The hardness need not immediately decrease following the beginning of the application. However, at a point in time appropriate to the application, a reduced hardness of the material is reached.

A preferred material of variable hardness is a composite material which contains two or more materials of which at least one material, preferably the hardest material, shows a decrease in hardness during application and/or is at least partially dissolved out. The composite material in accordance with the invention is broadly defined and includes all materials which can be obtained by combining different materials. These can be particulate, fibrous and/or layered composites. A material which is composed of different portions, strips or layers of material is also to be regarded as a composite material in accordance with this invention. The initial composition of the composite material is selected such that the cannula exhibits as high a penetrative capacity as possible in its initial state.

If the hardness of a material component of the composite material is changed, then the cannula pliability of the cannula as a whole—or in areas if the composite material in question is only arranged in areas—is changed on two levels. On the one hand, the hardness of this material component of the composite material is reduced, on the other hand this also leads to a decrease in hardness of the composite material as a whole, which for its part in turn represents a material component of the cannula or even forms the cannula as such.

If, in accordance with another preferred embodiment, a material component of the composite material is at least partially dissolved out, then its composition changes and the resultant hardness decreases during application. A material can be dissolved out either on an atomic or molecular level, or larger particles of material may be removed. Preferably, the hardest material is removed, however in accordance with the invention a number of materials having any hardness can also be removed, providing dissolving them out generates a composite material having a reduced hardness, during application.

In some embodiments, a dissolved out material is preferably a bio-compatible material.

In accordance with a preferred embodiment, the composite material contains a solid state material and an organic polymer or is formed by this combination of materials alone. Any solid state material may be used which can be processed together with other material components, in particular one or more organic polymers, into a composite material. It is preferably an inorganic solid state material.

In another preferred embodiment, the material of variable hardness is or comprises a water-absorbing material, preferably a water-absorbing polymer. Water may diffuse into the material during application from the outer side of the cannula, the inner side of the cannula or from both sides. If the material is a polymer, then the water absorption weakens the interaction between the polymer chains, which manifests itself in a decrease in hardness. The water which has diffused into the polymer structure therefore functions as a plasticizer. In some embodiments, the material is preferably a polymer based on a polyamide.

In a preferred embodiment, the cannula in accordance with the invention consists only of material of variable hardness. In another preferred embodiment, the cannula additionally contains a material which exhibits a lower hardness, prior to application or use of the cannula, than the material of variable hardness. In this case, either the material of variable hardness at least partially surrounds the material having a lower initial hardness or vice versa. In accordance with the invention, the materials can be held together at their interface by strong interactions or can be moved slightly against each other due to weak interactions. Furthermore, one material can be applied in the form of a coating to the other material.

The material having a lower initial hardness is preferably a material which does not change in its hardness during application. It is thus conceivable in accordance with the invention that, due to the decrease in hardness of the material of variable hardness during application, this material falls below the initial hardness of the other material.

In preferred embodiments of the cannula in accordance with the invention, the material having the greater hardness, which is at least partially yielded during application, at least partially surrounds the material having the lower hardness, or vice versa. The material having the greater hardness can be yielded in any way (as used herein, the term “yield” and its variants are intended to mean given up, used, used up, eliminated, dissolved, reduced and the like), for example by being mechanically removed or dissolved away. The material can be dissolved away, during application, at the atomic or molecular level or in the form of larger particles. Dissolving the material is also to be understood by this. This also applies with respect to all the other embodiments in which the terms “yield,” “yielded,” “dissolved” or “dissolving away” is used. In some embodiments, the material dissolved is preferably a bio-compatible, absorbable and/or bio-absorbable material or the like.

As already mentioned, the rigidity of the cannula in accordance with the invention in its initial state enables the skin or a septum to be penetrated without problems, while the increasing pliability in the course of it use enables the cannula to be flexibly moved, guided and/or adjusted. In some preferred embodiments, the increase in pliability is completed within five hours, in others, preferably within two hours and particularly preferably within one hour, following the beginning of use. In accordance with the invention, the increase in pliability can be concluded after 60 seconds at most.

The cannulae in accordance with the invention can be used in a transcutaneous infusion set, a transcutaneous perfusion set and/or a catheter head for one of the above apparatus.

In accordance with the invention, a transcutaneous infusion set is also provided which includes a cannula in accordance with the invention. This enables the cannula to be specifically positioned during application due to its pliability and flexibility, irrespective of the insertion point selected. This can be achieved by guiding the cannula inside the vascular system to a target location. The fluid to be infused can thus be better conveyed to its desired effective location.

In accordance with another embodiment, a transcutaneous perfusion set is provided which includes a cannula in accordance with the invention. Analogously to the infusion set, the cannula can be better conveyed to a desired target location in order to there remove body fluid by perfusion.

In accordance with the invention, a catheter head for a transcutaneous infusion set or perfusion set is furthermore provided, wherein the cannula in accordance with the invention forms an infusing part of the catheter head. A cannula is used which, due to its pliability and flexibility in its initial state, can puncture both a septum within the catheter head and also the skin, each at an angle of 90°±20°, or any angle favorable for penetration, wherein the puncture planes formed by the skin and the septum or number of septa can be arranged an at angle to each other. Furthermore, the pliability and flexibility is such that, due to its property in accordance with the invention, the cannula in the body is not perceived as bothersome or, preferably, is not perceived at all. The rigidity with respect to penetration can be entirely comparable to that of a steel needle. It is not necessary to employ another cannula. A catheter head in accordance with the invention can, however, comprise other cannulae in accordance with the invention or additional conventional cannulae, which can fulfill other functions.

### BRIEF DESCRIPTION OF THE DRAWINGS

FIG. 1, including FIGS. 1a and b, depicts a cannula made of materials of differing hardness;

FIG. 2 depicts a cannula made of a composite material of variable hardness;

FIG. 3 depicts a cannula made of material of variable hardness;

FIG. 4 depicts a cannula with an outer material of variable hardness;

FIG. 5 depicts a cannula with an inner material of variable hardness;

FIG. 6 depicts a cannula made of a composite material of variable hardness;

FIG. 7a depicts a system of flexible cannulae of variable hardness; and

FIG. 7b depicts another system of flexible cannulae of variable hardness.

### DETAILED DESCRIPTION

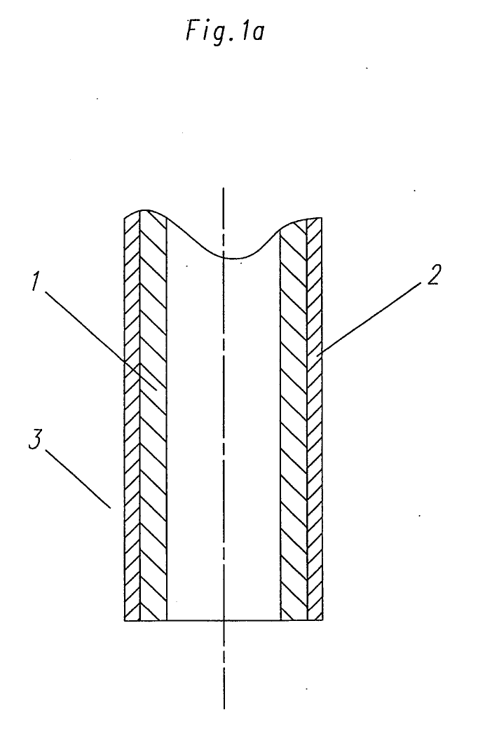

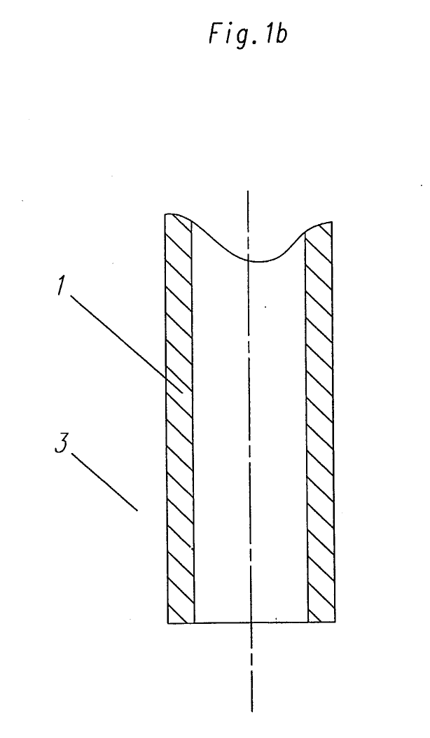

FIG. 1 shows a cannula (3) prior to application (FIG. 1a) and at a later point in time during application (FIG. 1b). Prior to application, the cannula consists of two materials of differing hardness, namely an inner core (1) and an outer material, wherein the latter exhibits a greater hardness than the inner core (1). This provides the cannula with the necessary rigidity for penetrating the skin, a septum or other materials. The harder material is absorbable material. This can for example be applied to the material having the lower hardness in the form of a coating. In accordance with the invention, the harder material can also be coated with the material having the lower hardness. Another possibility is to produce the core and outer material separately, with different inner diameters, in order to then assemble them into the cannula in accordance with the invention.

During application, the material having the greater hardness is dissolved away, such that eventually a cannula in accordance with FIG. 1b is created. In the present example, the material is completely dissolved away. As the case may be, however, it may be sufficient to only partially dissolve away the coating. Following the beginning of application, a decrease in the pliability is caused within one hour, which enables the cannula to be used in accordance with the invention in a transfusion set or a perfusion apparatus.

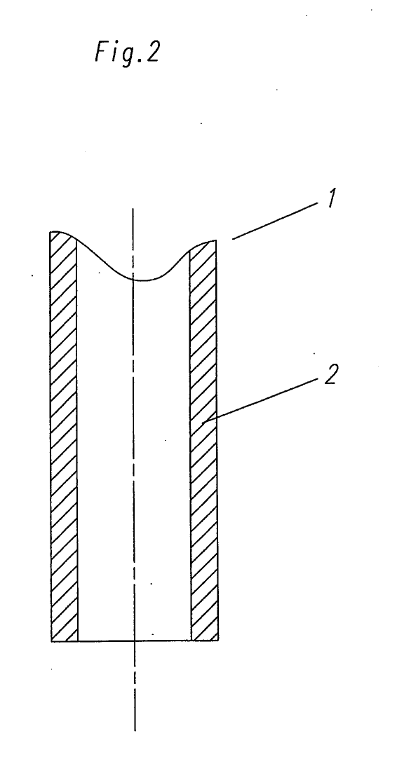

FIG. 2 shows a cannula (1) made of a composite material (2) whose hardness decreases during application. The composite material consists of at least two materials of differing hardness. Following the beginning of application, a material—or a number of materials, as the case may be—are at least partially dissolved away from the cannula. Preferably, the material having the greatest hardness is yielded. This process can for example be triggered by the cannula coming into contact with body fluid, by contacting it with infusion or perfusion solutions, or by other methods.

Due to the dissolving away, the cannula created during application has a changed material composition and as a consequence also an increased pliability. Removing a material from the composite material also leads, as the case may be, to structural changes such as can generate a permeability of the cannula to fluid, at least in sections. Following the beginning of application, a decrease in the pliability is caused within one hour, which enables the cannula to be used in accordance with the invention in a transfusion set or a perfusion apparatus.

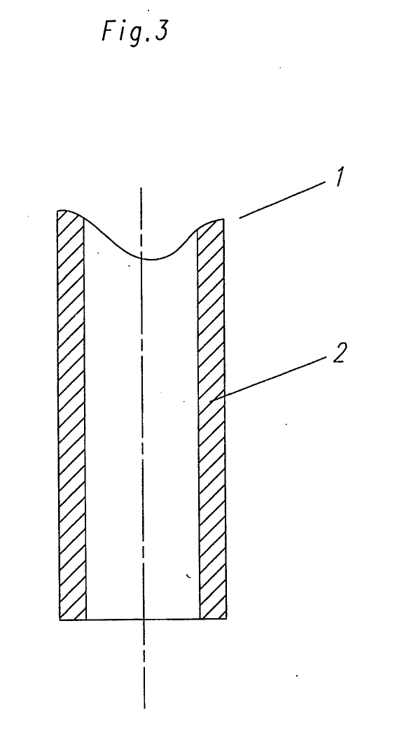

FIG. 3 shows a cannula (1) made of a material (2) whose hardness changes during application. This can be, for example, a water-absorbing material whose hardness decreases due to an absorption process. Suitable materials are, for example, polymers comprising polar functional groups which enable water molecules to be absorbed, and other suitable materials are envisioned as well. In the instance of polymers comprising polar functional groups, thabsorption process reduces the interactions between the polymer chains and leads to a reduction in the hardness of the material. The water molecules function to a certain extent as “plasticizers.” Water may be absorbed both from the inner side of the cannula and the outer side of the cannula.

The cannula material in FIG. 3 can also be a material which changes its hardness under the influence of temperature. Following application into the tissue, for example, the influence of body temperature could reduce the hardness. Suitable exemplary materials are polymers or polymer mixtures whose mechanical properties are influenced by changes in temperature. Polymer mixtures may be used which contain polymers whose glass transition temperature is selected such that an application at body temperature alone leads to a sufficient decrease in the hardness of the material.

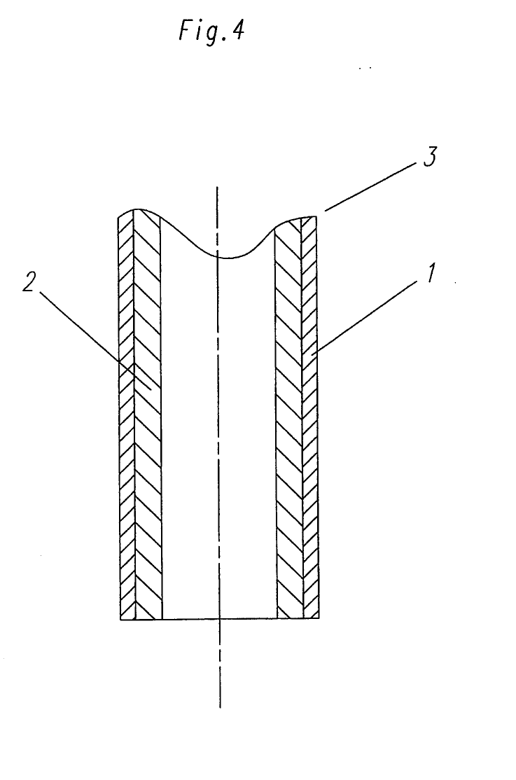

FIG. 4 shows a cannula made of an outer material (1) of variable hardness and an inner material (2) having a lower initial hardness, i.e., prior to application, the outer material (1) exhibits a greater hardness than the inner material (2). The outer material (2) can for example be produced by surface modification, coating or coextrusion.

The material of variable hardness can be a composite material as described in FIG. 2, or also a material as described in FIG. 3. If the outer material includes a water-absorbing polymer, then water is absorbed from the outer side of the cannula.

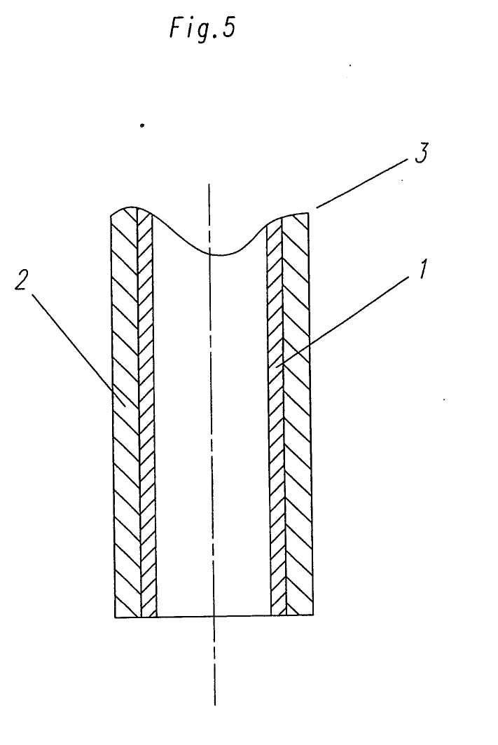

FIG. 5 shows a cannula (3) which is derived from the cannula in FIG. 4, with the difference that in this embodiment, the material (1) of variable hardness is surrounded by a material (2) having a lower initial hardness. If the material of variable hardness is a water-absorbing polymer, then water is absorbed through an inner area of the cannula, e.g., its inside wall or a portion thereof, when infusion solutions are transported through the cannula during application.

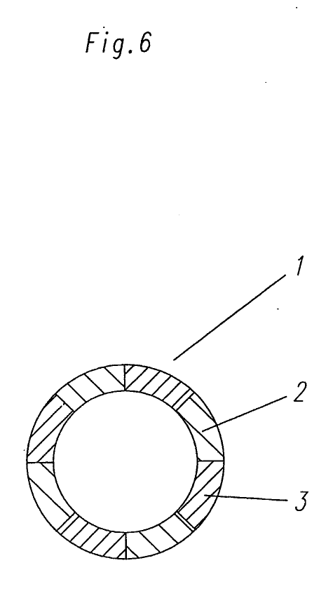

FIG. 6 shows a cannula (1) made of a composite material formed by strips of a material of invariable hardness (2) and strips of a material of variable hardness (3) which are arranged adjacent to each other in the circumferential direction. During application of the cannula (1), the hardness of the strips of the material (3), and therefore also of the composite material, decreases, which leads to a cannula which increases in pliability during application.

The strips of the material (2), (3) can each extend over the whole length of the cannula or can be provided only in sections. The width of the strips and the number of strips can vary in any way and can be adjusted to the desired properties of the composite material consisting of said strips. The strips (2), (3), having different hardness properties, in one embodiment preferably form the cannula casing in an alternating and uniform distribution.

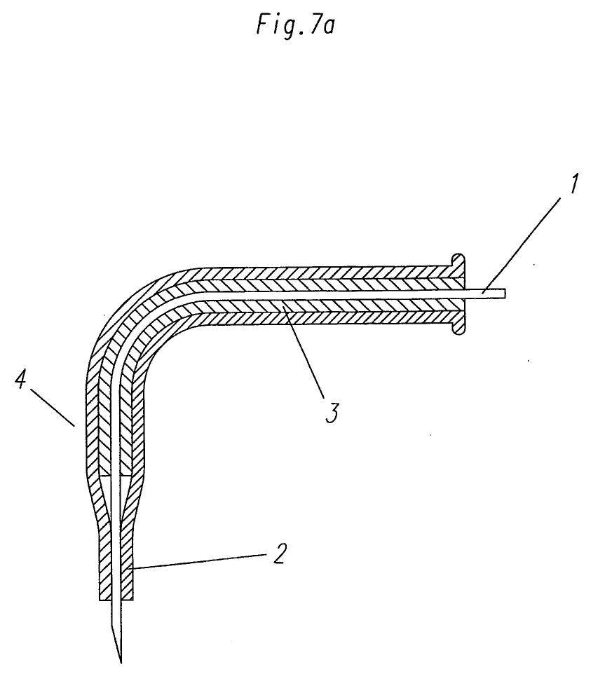

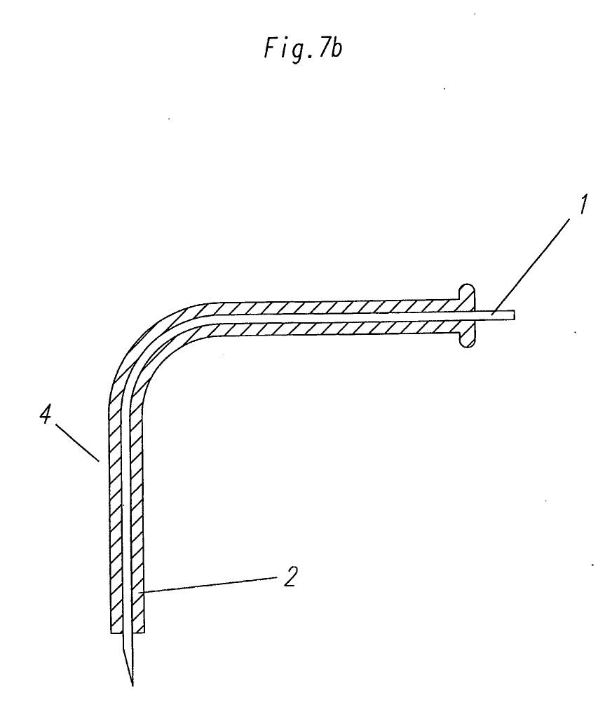

FIG. 7 shows a flexible cannula (4) comprising two separate, equally flexible cannulae (1), (2), wherein cannula (1) consists of a material having a greater hardness. Even in its initial state, however, cannula (1) exhibits a pliability which enables the cannula (4) to be bent with a radius of curvature of preferably less than 5 cm and to penetrate the skin, a septum or other materials without problems. The pliancy is preferably sufficiently great that a radius of curvature of 0.5 cm or less can be achieved. Particularly preferably, in one embodiment, the combination of the two cannulae (1) and (2) can be bent up to a radius of curvature of 0.1 cm or less. In one embodiment, cannula (1) is preferably a metallic needle, for example a hollow needle made of steel, with an outer diameter preferably in the range of 0.1 mm to 0.3 mm. Due to the material having a lower initial hardness, cannula (2) offers no resistance or only slight resistance to a bending movement, and adjusts to the shape of the flexible cannula (1). Cannula (1) can also be replaced by a needle of solid material.

During application of the cannula (4), cannula (1) is removed, such that only cannula (2) remains in the penetrated material, e.g., in the body of the patient. This leads to an increase in pliability in accordance with the invention.

As shown in FIG. 7a, the flexible cannula (4) can, as the case may be, comprise a protecting device (3), so that the cannula (2) is not damaged when the cannula (1) is removed. The protecting device (3) consists of a material whose hardness guarantees sufficient protection for the cannula (2) and whose pliability does not compromise the flexibility of the resultant cannula once the cannula (1) has been removed. The protecting device offers only slight resistance when the cannula (1) is removed, such that this process can be performed without problems. FIG. 7b shows a cannula (4) in accordance with the invention, without a protecting device.

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

What is claimed is:

1. A cannula that increases in pliability during use, wherein the cannula comprises a composite material formed by strips of material of an invariable hardness and by strips of a material of variable hardness, said strips of invariable and variable hardness arranged adjacent to each other in the circumferential direction, wherein, when the cannula is in use, the material of variable hardness decreases in hardness.

2. The cannula as set forth in claim 1, wherein the strips of invariable and variable hardness are arranged in an alternating configuration.

3. The cannula as set forth in claim 1, wherein the strips of invariable and variable hardness are arranged along a length of the cannula.

4. The cannula as set forth in claim 1, wherein the strips of invariable and variable hardness are arranged along one or more sections of the cannula.

5. A cannula that increases in pliability during use, wherein the cannula comprises a water-absorbing material based on a polyamide of a first variable hardness that decreases in hardness upon water absorption, and a material having a second hardness that at least partially dissolves upon use.

6. The cannula as set forth in claim 5, wherein when said cannula is in use, said cannula comprises a water absorbing inner side and a water absorbing outer side.

7. The cannula as set forth in claim 5, wherein said water-absorbing material based on a polyamide of a first variable hardness comprises an inner material of said cannula, and said material having a second hardness comprises an outer material of said cannula.

8. A cannula which increases its pliability during use, wherein, prior to application, said cannula comprises at least two materials having a different hardness of which said material having the greater hardness is yielded during use, wherein said cannula has a bent shape.

9. The cannula as set forth in claim 8, wherein the cannula has a curved shape.

10. The cannula as set forth in claim 9, wherein the curved cannula comprises a radius of curvature of less than 5 cm.

11. The cannula as set forth in claim 10, wherein the radius of curvature is less than 0.5 cm.

12. The cannula as set forth in claim 10, wherein the radius of curvature is less than 0.1 cm.

13. The cannula as set forth in claim 8, wherein the material having the greater hardness is a second cannula, said second cannula being removed during use.

14. The cannula as set forth in claim 13, wherein the cannula with the greater hardness is a metallic needle, said metallic needle having an outer diameter in the range of 0.1 mm to 0.3 mm.

15. The cannula as set forth in claim 8, wherein the materials of differing hardness are separated by a layer, at least partially in sections.

16. The cannula as set forth in claim 8, wherein the material having the lower hardness comprises a material having a hardness that can vary during use.

17. The cannula as set forth in claim 8, wherein said cannula is configured in a transcutaneous perfusion set.

18. The cannula as set forth in claim 8, wherein said cannula is configured in a catheter head for a transcutaneous infusion set, in which the cannula forms an infusing part of said catheter head.

19. A cannula which increases in pliability during use, wherein, prior to application, said cannula comprises a first material of a first thermally susceptible hardness and a second material having a second hardness, that, prior to use is lower than the harness of the first material, wherein, the thermally susceptible hardness of said first material decreases during use, wherein, the first material is or comprises a polymer

20. The cannula as set forth in claim 19, wherein the second material having the lower initial hardness at least partially surrounds the material of the first variable hardness.

21. The cannula as set forth in claim 19, wherein the material having the lower hardness at least partially surrounds the material having the greater hardness.
